(19) United States
(12) Patent Application Publication
(10) Pub. No.: US 2016/0186167 A1
(43) Pub. Date: Jun. 30, 2016

(54) Method and Device for Processing a Sample of Biological Material Containing Target Cells and Companion Cells in Order to Extract Nucleic Acids of the Target Cells

(71) Applicant: Robert Bosch GmbH, Stuttgart (DE)

(72) Inventors: Bernd Faltin, Gerlingen (DE); Franz Laermer, Weil Der Stadt (DE); Sebastian Berning, Stuttgart (DE); Christian Dorrer, Winnenden (DE)

(21) Appl. No.: 14/910,543

(22) PCT Filed: Jul. 24, 2014
(86) PCT No.: PCT/EP2014/065883

§ 371 Date: Feb. 5, 2016

(30) Foreign Application Priority Data
Aug. 7, 2013 (DE) 10 2013 215 575.1

(51) Int. Cl. C12N 15/10; B01D 39/06; C12N 1/06

(52) U.S. Cl. CPC C12N 15/1017; C12N 1/06; B01D 39/06

(57) ABSTRACT

A method for processing a sample of biological material containing target cells and companion cells in order to extract nucleic acids of the target cells includes accumulating the target cells of the sample by separating the target cells or the companion cells from the sample. The method additionally includes decomposing the target cells by chemical or physical lysis in order to produce a target cell lysate containing the nucleic acids of the target cells. The method further includes purifying the nucleic acids from the target cell lysate in order to extract the nucleic acid of the target cells.

### PRIOR ART

The present invention concerns a method for processing a sample of biological material containing target cells and companion cells in order to extract nucleic acids of the target cells and a device for processing a sample of biological material containing target cells and companion cells in order to extract nucleic acids of the target cells. In particular, the present invention concerns the field of microfluidic systems, for example for the so-called chip laboratory or pocket laboratory (lab on a chip).

It is often necessary in molecular diagnosis to detect pathogenic DNA or RNA in a sample. ‘Pathogenic DNA or RNA’ refers to DNA or RNA obtained from a pathogen, e.g. a virus or a microbe such as a bacterium or fungus. ‘The sample’ is understood to refer in particular to a blood sample, but in principle, this may also mean another liquid or liquefied patient samples such as urine, stool, sputum, CSF, lavage fluid, washed-out smears, or liquefied tissue samples, particularly if they contain blood or traces of blood. Diseases for which this method is relevant also include sepsis, for example. In cases of suspected sepsis, it is important to detect pathogens in blood, and if applicable resistance to certain antibiotics. Because of the differing concentration ratios of pathogens to leucocytes, for example 10 to 1000 per mL as compared with 10⁶ to 10⁷ per mL, the human DNA background content of the sample in this case is quite high. Commercially available methods for the selective purification of pathogenic DNA, e.g. from blood, use chemical reagents, for example, in order to first achieve selective lysis of human cells. After this, the human nucleic acids are enzymatically digested. Pathogens are subsequently isolated from the supernatant, for example by centrifugation and decanting. This type of method is disclosed, for example, in DE 102005009479 A1.

US 2010/0285578 A1 discloses devices and a method for obtaining nucleic acids from biological samples.

### DISCLOSURE OF THE INVENTION

Against this backdrop, an improved method for processing a sample of biological material containing target cells and companion cells in order to extract nucleic acids of the target cells and an improved device for processing a sample of biological material containing target cells and companion cells in order to extract nucleic acids of the target cells according to the main claims is presented. Advantageous embodiments are disclosed in the respective sub-claims and the following description.

According to embodiments of the present invention, preparation of a biological sample, specifically for selectively obtaining pathogenic nucleic acids from a sample not containing pathogenic nucleic acids, for example from blood cells, can be carried out. Embodiments of the present invention comprise e.g. a concept for the thermal pretreatment of a sample and/or a concept for the preparation of a sample by means of a membrane, a microduct structure, or the like. Specifically, this makes it possible to achieve a combination of thermal pretreatment in which companion cells are selectively lysed by enzymatic digestion and separation by means of filtration for the purpose of obtaining purified target cell nucleic acids from a sample that also contains companion cells. In this case, separation of leucocytes, e.g. with human DNA from a blood sample, that are to be tested for the presence of pathogens, is possible.

Embodiments of the present invention can be used in a particularly advantageous manner in systems or laboratory routines used in molecular diagnosis or in microfluidic lab on chip systems for molecular diagnosis.

Preferably, embodiments of the present invention make it possible to reliably separate target cell nucleic acids, e.g. pathogenic DNA, contained in a sample from companion cell nucleic acids, e.g. human DNA. This prevents the companion cell nucleic acids from interfering with the subsequent amplification and detection steps, and the sensitivity of detection or diagnosis is thus improved. In particular, in thermal pretreatment of a sample, one can use methods such as enzymatic digestion to reliably prevent the sample from gelling, which would occur, for example, if the temperature and/or the duration of pretreatment is too long. In this case, gelling of the sample can also be prevented in light of the fact that the critical temperature and duration with respect to gelling also depend on characteristics of the sample such as hematocrit. For example, enzymatic digestion can be carried out in order to prevent the sample from clogging a filter. This also makes it possible to process large sample amounts. In this case, filtration is carried out in order to accumulate a small number of pathogens, e.g. 10 to 1000, from a relatively large volume of blood, e.g. 1 to 10 μL. This makes it possible to increase the effective concentration of target cell nucleic acids and facilitates subsequent amplification and detection.

Embodiments of the present invention are particularly well-suited for automation, particularly in a microfluidic system. This can facilitate the process and reduce the risk of contamination.

According to embodiments of the present invention, reliable isolation and concentration of target cell nucleic acids from a sample can be achieved. This allows the efficiency of subsequent amplifications and/or detection steps to be improved. Separation of the companion cells according to the invention is preferred to chemically selective lysis of the companion cells in that the number of reagents and work steps required can be minimized. This also allows the time required for sample preparation to be reduced. There is also an advantage with respect to purification of target cell nucleic acids in that embodiments of the present invention can be simply integrated into a microfluidic system, e.g. a lab on chip system (LOC) for molecular diagnosis. The degree of separation efficiency of target cell nucleic acids with respect to companion cell nucleic acids can also be increased, and the number of required filters or membranes can be minimized. In particular, the filtration process can be based on differences in typical cell sizes, and is therefore universally applicable. Therefore, the need for adaptation to specific target cells characteristic of antibody-based methods, for example, is obviated.

A method for processing a sample of biological material containing target cells and companion cells in order to extract nucleic acids of the target cells comprises the following steps:

accumulating the target cells of the sample by separating the target cells or the companion cells from the sample; decomposing the target cells by chemical and/or physical lysis in order to produce a target cell lysate containing the nucleic acids of the target cells; and purification of the nucleic acids from the target cell lysate in order to extract nucleic acids of the target cells.

The target cells may comprise pathogenic cells or pathogens, e.g. viruses or microbes such as bacteria or fungi, that contain DNA and/or RNA as their nucleic acids. For purposes of simplicity, the term ‘nucleic acids’ will frequently be used in the following, with said term referring to DNA and/or RNA. The companion cells may comprise human cells such as blood cells or the like. In particular, ‘the sample’ can be understood to refer to a blood sample, or it can also be understood to refer to other liquid or liquefied patient samples, e.g. urine, stool, sputum, CSF, lavage fluid, washed-out smears, or liquefied tissue samples, particularly if they contain blood or traces of blood. In the purification step, the nucleic acids contained in the target cell lysate can be purified and then subjected to analysis in order to test for the presence of specified pathogens or genes, e.g. resistance genes. This subsequent analysis can be carried out e.g. by sequencing, the polymerase chain reaction (PCR), real-time PCR, and/or detection or hybridization on a microarray.

Purification of the target cell nucleic acids from the sample can be carried out after decomposition or lysis of the target cells by means of subsequent adsorption of the target cell nucleic acids to a solid phase, such as a silica filter, microparticles, or so-called beads. In addition to chemical and enzymatic lysis methods, there are also mechanical methods using e.g. ultrasound, microspheres, or beads. The purpose of purification is to make the target cell nucleic acids available in concentrated form for subsequent amplification and/or detection. Embodiments of the present invention can also be advantageously used in separating the target cell nucleic acids from cell debris and proteins. Human blood samples in particular, however, can also contain large amounts of companion cells with human DNA. One possibility for separating the target cells from the companion cells is to first selectively decompose or lyse the companion cells, e.g. blood cells, contained in the sample and separate them from the target cells. After this, the target cells can be lysed and the target cell nucleic acids can be purified. Embodiments of the present invention make it possible to separate the target cell nucleic acids from the background companion cell nucleic acids present in the sample. These companion cell nucleic acids would otherwise interfere with subsequent amplification and analysis of the target cell nucleic acids, potentially making it difficult to impossible to detect the presence of the target cells. In this manner, the companion cell nucleic acids can be prevented from forming undesired byproducts in subsequent amplification and testing, causing a reduction in sensitivity.

In a corresponding method, the accumulation step can include a partial step of tempering the sample to a lysis temperature for decomposition and pre-damaging the companion cells and a partial step of separating the target cells from the sample by filtration.

According to an embodiment, the accumulation step can include a partial step of tempering the sample to lysis temperature in order to decompose and pre-damage the companion cells, a partial step of lysing the companion cells pre-damaged in the partial tempering step by chemical or enzymatic lysis and digestion of nucleic acids released from the companion cells by enzymes, and a partial step of separating the target cells from the sample by filtration. In this case, a suitable lysis temperature may be selected such that the companion cells contained in the sample are destroyed or pre-damaged but the target cells contained in the sample remain intact. In the partial separation step, the target cells can be retained by an accumulation filter. Such an embodiment is advantageous in that particularly effective and reliable accumulation of the target cells can be achieved. In this case, thermal treatment can be applied selectively based on differences in the nature of the cell wall. Lysing can prevent clogging during filtration.

In this case, the partial step of chemical or enzymatic lysis and enzymatic digestion can take place before, during, or after the partial tempering step. This allows the viscosity of the sample to be reduced in the partial lysis step. In this case in particular, temperature-resistant enzymes can be used in the partial lysis and digestion steps, such as nucleases, proteinases, or lysozyme. Such an embodiment is advantageous in that it makes it possible to reliably prevent gelling of the sample as early as during thermal pretreatment.

Alternatively, the companion cells can be separated from the sample in the accumulation step by filtering them at least once. In this case, the companion cells can be retained by means of a separation device, which can comprise a filter, a membrane, microstructures or the like. In this case, separation takes place due to the differing size of the target and companion cells, i.e., the companion cells are retained if they are larger than the target cells. Such an embodiment is advantageous in that the companion cells can be removed from the sample at an early stage of the method.

In this case, the sample for separating the companion cells can be flushed through a plurality of separation devices connected in series in the accumulation step. Such an embodiment is advantageous in that multiple filtration allows separation efficiency to be further increased.

In this way, the number of companion cells contained in the filtrate, and thus the amount of undesirable nucleic acids, can be further reduced.

In the accumulation step, the sample for separating the target cells can be flushed through a separation device multiple times. The target cells are retained on the filter because of their size. In particular, this allows the target cells to be separated from companion cells lysed or pre-damaged in the partial lysis step. This in turn allows the device to be purified of companion cells between flushing operations. Cleaning can be carried out by flushing water or a buffer through the separation device in a direction opposite to that of the filtration device. Such an embodiment is advantageous in that the structure is simplified and only one separation device is required. By cleaning or washing the separation device, allowing the companion cells to be separated from the separation device, the flow resistance of the separation device can be reduced so that the subsequent filtration can be carried out in a simpler and faster manner, i.e. at lower pressures and higher flow rates. Moreover, this also makes it possible to prevent companion cells from detaching from the separation device and getting into the filtrate. The achievable separation efficiency is therefore increased.

According to an embodiment, the accumulation step can include a partial sample dilution step. In this step, the sample can be diluted with water or an aqueous buffer. Such an embodiment is advantageous in that the viscosity of the sample is reduced, thus improving the processability of the sample. If the accumulation step includes the partial step of tempering the sample, the partial dilution step can be carried out before or after the partial tempering step. On the one hand, this allows gelling during thermal treatment to be more reliably prevented, and on the other hand, it makes it possible to effectively carry out further lysis of companion cells pre-damaged during thermal treatment by means of osmotic shock.

In particular, in the decomposition step, the target cells can be decomposed by means of a lysis buffer, and additionally or alternatively by means of ultrasound coupling. Such an embodiment is advantageous in that fewer reagents are required, obviating the need for addition of a lysis buffer to decompose the target cells. Pressure waves and cavitation caused by the ultrasound allow the cell walls of the target cells to be destroyed in a particularly reliable and rapid manner.

A device for processing a sample of biological material containing target cells and companion cells in order to extract nucleic acids of the target cells comprises the following characteristic components:

a device for accumulating the target cells of the sample by separating the target cells or the companion cells from the sample;

a device for decomposing the target cells by chemical and/or physical lysis in order to produce a target cell lysate containing the nucleic acids of the target cells; and

a device for purifying the nucleic acids from the target cell lysate in order to extract nucleic acids of the target cells.

The above-described processing device can be advantageously applied or used in combination with an embodiment of the processing method to prepare a sample of biological material containing target cells and companion cells in order to allow extraction of the nucleic acids of the target cells. The device is configured so as to carry out or implement the steps of the processing method in relevant devices. By means of this modified embodiment of the invention in the form of a device, the object of the invention can be rapidly and efficiently achieved. The device can be configured as a microfluidic system, in particular for a so-called chip laboratory, pocket laboratory, or lab on a chip.

In a corresponding device, the accumulation device can comprise tempering means for tempering the sample to a lysis temperature in order to decompose or pre-damage the companion cells.

According to an embodiment, the accumulation device can comprise tempering means for tempering the sample to lysis temperature in order to decompose or pre-damage the companion cells, a storage chamber for a buffer solution used to lyse the companion cells decomposed or pre-damaged in the tempering step by chemical or enzymatic lysis, and an accumulation filter for separating the target cells from the sample. In this case, the tempering means can comprise a heating device or a heating device and a cooling device. The tempering means may also be configured for heating and/or cooling. Such an embodiment is advantageous in that particularly effective and reliable selection of the target cells is achieved. In this case, thermal treatment can be selectively applied based on differences in the nature of the cell wall. This lysis can prevent clogging during filtration. In particular, the tempering means makes it possible to precisely set the selection temperature. If the tempering means also comprises a cooling device, the sample can be cooled before lysis to a second specified temperature level such as room temperature, so that the duration of thermal pretreatment can be set with particular precision and gelling of the sample can also be prevented in a particularly reliable manner.

In this case, the tempering means can be thermally coupled with a sample chamber or with a sample duct arranged between a sample chamber and a decomposition chamber. Such an embodiment is advantageous in that thermal treatment of the sample can take place, according to the application, in a stationary manner or according to the flow-through principle.

In addition, the accumulation filter of the accumulation device can also be used by the decomposition device, and additionally or alternatively, the purification device.

Such an embodiment is advantageous in that a filter is not needed when the target cells are lysed on the accumulation filter, and the accumulation filter is also used for DNA purification. In order to achieve this, the lysis buffer used for decomposition can be configured in such a way that the target cell nucleic acids released during decomposition bind directly to the accumulation filter. Alternatively, following decomposition, a binding buffer can be added to the accumulation filter without displacing the lysis buffer from the accumulation filter. In this case, mixing of the lysis buffer and binding buffer in the accumulation filter can be carried out by diffusion.

Alternatively, the accumulation device can comprise at least one separation device for separating the companion cells from the sample. In this case, the at least one separation device can comprise a separation filter, a filter membrane, a filter duct having a plurality of integrated columns or posts, and additionally or alternatively, a section provided with filter pores. Such an embodiment is advantageous in that the companion cells can be reliably separated from the sample at an early stage of the method. If the at least one separation device has sieve-like microstructures configured in the channels and chambers of a microfluidic system, and in particular can be manufactured in the same production step as other structures of the microfluidic system, this simplifies manufacturing of the device, as separate steps for integrating a separate separation device are not necessary. Alternatively, membranes having a specified pore diameter can also be molded by means of such a method directly in the bottom of a microfluidic duct or a chamber.

The separation device and a return duct can also be configured in parallel between the sample chamber and the decomposition chamber. In this case, a washing device or flushing device for cleaning the separation device to remove filtered-out companion cells can also be provided. Such an embodiment is advantageous in that the sample for separating the companion cells can be flushed several times through the separation device, allowing separation efficiency to be increased. The device for cleaning the separation device further facilitates separation by reducing the flow resistance of the device.

Moreover, the accumulation device can comprise a plurality of separation devices connected in series between the sample chamber and the decomposition chamber. In this case, the separation devices may be the same or different. Such an embodiment is advantageous in that separation efficiency can be further increased by multiple filtration.

The invention is explained by way of example with reference to the attached drawings. Specifically:

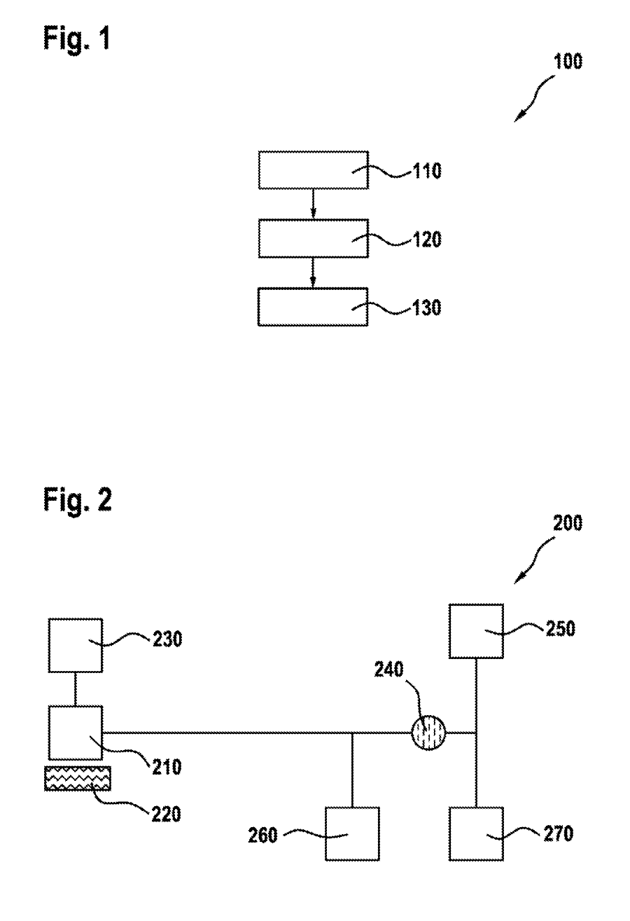

FIG. 1 shows a flow chart of a method for processing according to an exemplary embodiment of the present invention; and

FIGS. 2 to 15 show devices for processing according to exemplary embodiments of the present invention.

In the following description of preferred exemplary embodiments of the present invention, identical or similar reference numbers are used for the elements shown in the various figures and having similar actions, with a repeated description of these elements being dispensed with.

FIG. 1 shows a flow chart of a method 100 for processing a sample of biological material containing target cells and companion cells in order to extract nucleic acids of the target cells according to an exemplary embodiment of the present invention. The method 100 for processing is advantageously conducted by connection to a device such as the device for processing of one of FIGS. 2 to 15. The method 100 has a step 110 of accumulating the target cells of the sample by separating the target cells or the companion cells from the sample. The method 100 also has a step 120 of decomposing the target cells by chemical and, additionally or alternatively, physical lysis in order to produce a target cell lysate containing the nucleic acids of the target cells. Moreover, the method 100 comprises a step 130 of purifying the nucleic acids from the target cell lysate in order to extract nucleic acids of the target cells.

According to an exemplary embodiment, the accumulation step 110 includes a partial step of tempering the sample to lysis temperature for decomposition of the companion cells, a partial step of lysing the companion cells by chemical or enzymatic lysis and digesting the nucleic acids released from the companion cells by enzymes, and a partial step of separating the target cells of the sample by means of filtration. In this case, according to an exemplary embodiment, the partial lysis step and the digestion step can take place before, during, or after the partial tempering step, with the viscosity of the sample being reduced in the partial lysis and digestion steps.

According to an exemplary embodiment, the companion cells are separated from the sample by being filtered at least once in the accumulation step 110.

According to an exemplary embodiment, the sample for separating the companion cells is flushed through a plurality of separation devices connected in series in the accumulation step 110. Alternatively, according to an exemplary embodiment, the sample for separating the companion cells is flushed several times through a separation device in the accumulation step 110. In this case, the separation device can be cleaned to remove companion cells between flushing steps.

According to an exemplary embodiment, the accumulation step 110 includes a partial sample decomposition step.

In the decomposition step 120, according to an exemplary embodiment, the target cells are decomposed by means of a lysis buffer, and additionally or alternatively by means of ultrasound coupling.

FIG. 2 shows a device 200 for processing a sample of biological material containing target cells and companion cells in order to extract nucleic acids of the target cells according to an exemplary embodiment of the present invention. In this case, the device 200 is configured as a microfluidic system or a component of a microfluidic system. The components of the device 200 are shown here as a sample chamber 210, a tempering device in the form of a heater 220, a storage chamber 230 for a first buffer, a filter 240, a waste material chamber 250, a lysis buffer storage chamber 260, and a collection chamber 270.

The heater 220 is arranged adjacent to the sample chamber 210. In this case, the heater 220 is thermally coupled to the sample chamber 210. The storage chamber 230 is connected to the sample chamber 210 by means of a fluid connection. The filter 240 is arranged between the sample chamber 210 on the one hand and the waste material chamber 250 and collection chamber 270 on the other. In this case, the sample chamber 210 is connected by means of a fluid connection via the filter 240 to the waste material chamber 250 and the collection chamber 270. The lysis buffer storage chamber 260 is connected in a fluid-conducting manner to a fluid connection between the sample chamber 210 and the filter 240.

The sample chamber 210 comprises an opening for receiving the sample that can be reclosed e.g. by means of a stopper, a cover, or an adhesive film. For example, the heater 220 can be a Peltier heater or a film heater. The waste material chamber 250 is used to take up the filtrate. The collection chamber 270 is used to take up the lysate.

Thus, FIG. 2 shows a topology of the device 100 or a microfluidic system, wherein thermal treatment of the sample is carried out in a stationary manner in the sample chamber 210. Operation of the device 200 will be discussed below.

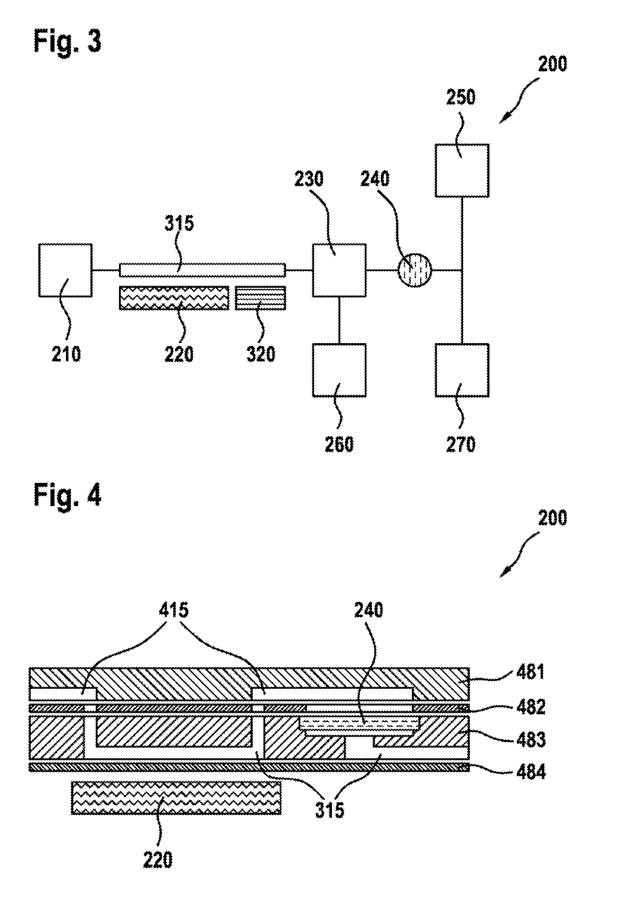

FIG. 3 shows a device 200 for processing a sample of biological material containing target cells and companion cells in order to extract nucleic acids of the target cells according to an exemplary embodiment of the present invention. The device 200 is similar to the device of

FIG. 2. The components of the device 200 shown are the sample chamber 210, the heater 220, the storage chamber 230 for the first buffer, the filter 240, the waste material chamber 250, the lysis buffer storage chamber 260, the collection chamber 270, the microduct 315, and a cooler 320.

The microduct 315 is arranged between the sample chamber 210 and the storage chamber 230. The sample chamber 210 is connected by means of a fluid connection via the microduct 315 to the storage chamber 230. The heater 220 and the cooler 320 constitute the tempering device. In this case, the heater 220 and the cooler 320 are arranged adjacent to the microduct 315. The heater 220 and the cooler 320 are thermally coupled to the microduct 315. In this case, the storage chamber 230 is arranged between the microduct 315 and the filter 240. The filter 240 is arranged between the storage chamber 230 on the one hand and the waste material chamber 250 and the collection chamber 270 on the other. The lysis buffer storage chamber 260 is connected by means of a fluid connection to the storage chamber 230.

Thus, FIG. 3 shows a topology of the device 200 for carrying out thermal treatment in flow-through mode. In this case, the sample from the sample chamber 210 is pumped through the temperable microduct 315 into the storage chamber 230 in which the first buffer is located. The temperable microduct 315 can be heated by the heater 220, and as needed, cooled by the cooler 320, e.g. a

Peltier cooler. By means of the cooler 320 as a second thermally active element, the heated sample can be tempered to a specified temperature level, e.g. room temperature, before being mixed with the first buffer. This is advantageous in that the duration of thermal pretreatment can be set with particular precision, and gelling of the sample can thus be prevented in a particularly reliable manner. Operation of the device 200 will be discussed below in further detail.

FIG. 4 shows a device 200 for processing a sample of biological material containing target cells and companion cells in order to extract nucleic acids of the target cells according to an exemplary embodiment of the present invention. The device 200 is similar to the device of FIG. 3, with FIG. 4 showing the device 200 in an embodiment as a multilayer structure in a sectional view and the chambers of the device 200 not being shown in the figure. The components of the device 200 shown are the heater 220, the filter 240, the microduct 315, a further microduct 415, a first structured polymer layer 481, a polymer film 482, a second structured polymer layer 483, and a polymer covering film 484.

The structured polymer layers 481 and 483 are molded for example from thermoplastic polymers, e.g. PP, PC, PE, PS, COP, COC, etc. The polymer film 482 is molded for example from thermoplastic polymers, thermoplastic elastomers, elastomers or the like. The covering film 484 comprises, for example, a thermoplastic film, an adhesive film, or the like.

The further microduct 415 extends between the temperable microduct 315 and the filter 240. The first structured polymer layer 481, the polymer film 482, the second structured polymer layer 483, and the polymer covering film 484 represent the multilayer structure of the device 200 according to the exemplary embodiment of the present invention shown in FIG. 4. Such an embodiment is advantageous in that it can be inexpensively manufactured and in that active elements such as valves can also be produced by means of the polymer film 482. Another advantage of this exemplary embodiment of the present invention is that the temperable microduct 315 is separated from the heater 220 only by the thin covering film 484. This allows the temperature in the temperable microduct 315 to be set with particular precision. Operation of the device 200 will be discussed below in further detail.

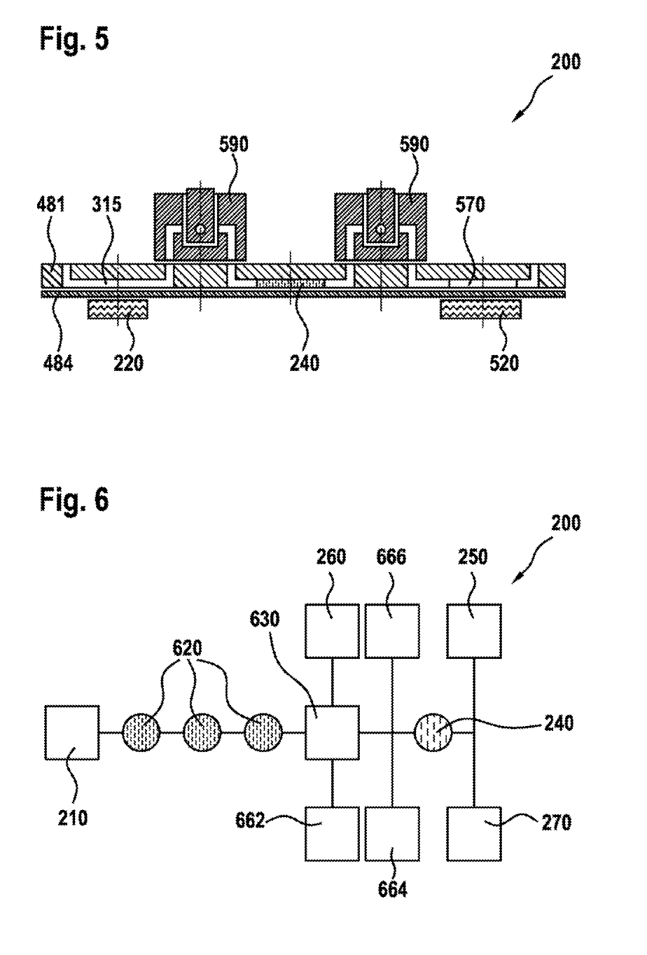

FIG. 5 shows a device 200 for processing a sample of biological material containing target cells and companion cells in order to extract nucleic acids of the target cells according to an exemplary embodiment of the present invention. The device 200 is similar to the device of FIG. 4. The components of the device 200 shown are the heater 220, the filter 240, the microduct 315, the first structured polymer layer 481, the polymer covering film 484, a further heater 520, a heatable amplification chamber 570, and as an example, two rotary valves 590.

In this case, the first structured polymer layer 481 and the polymer covering film 484 represent the multilayer structure of the device 200 according to the exemplary embodiment of the present invention shown in FIG. 5. The first of the rotary valves 590 is arranged between the microduct 315 and the filter 240 and configured so as to release or block a fluid connection between these two components. The second of the rotary valves 590 is arranged between the filter 240 and the amplification chamber 570 and configured so as to release or block a fluid connection between these two components. The further heater 520 is arranged adjacent to the amplification chamber 570 thermally coupled thereto. The filter 240 is therefore arranged between the two rotary valves 590.

Thus, FIG. 5 shows an embodiment of the device 200 as a multilayer structure with only two layers. Such an embodiment is particularly advantageous in that the device 200 can be inexpensively manufactured. The liquids are controlled in this case by the rotary valves 590. Moreover, such an embodiment also has an additional amplification chamber 570 that is heatable by means the further heater 520, in which, after purification of the target cell nucleic acids, amplification thereof by PCR can be carried out.

In the following, a concept for the thermal pretreatment of a sample using the method 100 and the device 200 according to exemplary embodiments of the present invention is presented with reference to FIGS. 1 to 5.

In the accumulation step 110, according to an exemplary embodiment, the actual thermal pretreatment of the sample is carried out in a first partial step. In this case, the sample is heated for example to a temperature or lysis temperature of 60 to 90° C., and preferably 65 to 85° C. In this case, the temperature is set in such a manner that the companion cells contained in the sample, e.g. blood cells such as leucocytes, are destroyed or pre-damaged and the nucleic acids contained in the companion cells, i.e. human DNA, are at least partially released, with the target cells contained in the sample, i.e. pathogens, remaining intact. Such selective lysis of the companion cells is possible because as pathogens, the target cells have a more robust cell wall, which makes them more stable with respect to thermal stresses. Heating of the sample can take place e.g. in stationary fashion in one of the sample chambers 210 or in flow-through mode in a capillary, a tube, or a duct such as the microduct 315. The liquid produced in this partial step is referred to as the first lysate.

In the accumulation step 110, a second partial step is carried out comprising mixing of the first lysate with a first buffer from the storage chamber 230 that contains enzymes, e.g. proteases, DNAses and lysozyme. This first buffer causes digestion or crushing of the damaged cells produced or released in the first partial step, as well as cell debris, proteins, and DNA strands of the companion cells. This digestion is essential for the subsequent partial filtration step, as gelling of the first lysate is prevented and the viscosity of the first lysate is reduced, thus preventing clogging of the filter 240. In this filtration, it is also possible to more simply separate still intact cellular components of the first lysate. The liquid produced in this partial step is referred to as digested lysate.

In the accumulation step 110, filtration then takes place in a third partial step, with the digested lysate being fed through the filter 240, e.g. a sterile, tissue, or silica filter. Still intact cellular components, in particular the target cells, are retained on the filter 240 because of their size and thus accumulate.

According to an exemplary embodiment, the sample is diluted with an aqueous buffer before the first partial step of the accumulation step 110, e.g. at a ratio of between 10:1 and 1:10. This is advantageous in that the viscosity of the sample is reduced and gelling during thermal treatment is more reliably prevented. If needed, dilution with the aqueous buffer can also be carried out after the first partial step of the accumulation step 110. This is advantageous in that osmotic shock resulting therefrom also makes it possible to effectively lyse companion cells that were only pre-damaged in the first partial step.

According to an exemplary embodiment, in the second partial step of the accumulation step 110, further components are added, e.g., detergents such as saponins, SDS or the like, chaotropic salts, or basic components such as NaOH. This is advantageous in that companion cells that were only pre-damaged in the first partial step of the accumulation step 110 are also efficiently lysed and their nucleic acids are released.

According to an exemplary embodiment, the first partial step of the accumulation step 110 is carried out after the second partial step of the accumulation step 110. This allows gelling of the sample to be reliably prevented as early as during thermal pretreatment. In this case, temperature-resistant enzymes are used in the first buffer.

According to an exemplary embodiment, instead of lysing the target cells on the filter 240 in the decomposition step 120, theses cells are flushed off the filter 240 before mixing with the lysis buffer, e.g. by flushing a buffer such as an aqueous buffer through the filter 240 in the opposite direction.

According to an exemplary embodiment, if the target cells in the decomposition step 120 are lysed on the filter 240, the filter 240 is preferably directly used in the purification step 130 as well. This is advantageous in that a filter is saved. In order to achieve this, the lysis buffer can be adjusted in such a way that the target cell nucleic acids released in the decomposition step 120 bind directly to the filter 240. Alternatively, following the decomposition step 120, a binding buffer can be added to the filter 240 without displacing the lysis buffer from the filter 240. Mixing of the lysis buffer and binding buffer in the filter 240 is then carried out by diffusion.

According to an exemplary embodiment, during the decomposition step 120, the digested lysate is also heated or subjected to ultrasound processing. Thermal stress or pressure waves and cavitation caused by the ultrasound make it possible to destroy the cell walls of the target cells in a particularly reliable and rapid manner.

A possible further method of processing the sample of biological material containing the target cells and companion cells in order to extract nucleic acids of the target cells is described in the following.

In the decomposition step 120, the target cells are lysed. This gives rise to a second lysate. Lysis or decomposition is carried out by adding the lysis buffer from the lysis buffer storage chamber 260 to the filter 240. This lysis buffer can contain enzymes such as proteinase K, proteases, and lysozyme. These enzymes destroy the cell walls of the target cells and therefore release the target cell nucleic acids. The cell wall of the target cells can also be destroyed in another manner, e.g. by adding chemical reagents such as chaotropic salts, detergents such as saponins, SDS or the like, β-mercaptoethanol, or basic components such as NaOH.

In the purification step 130, the target cell nucleic acids are purified from the second lysate, for example by adsorption to a solid phase.

Typically, the purification step 130 is followed by analysis of the target cell nucleic acids. For example, the purpose of this testing can be to detect the presence of specified pathogens and resistance genes.

Typically, for this purpose, the target cell nucleic acids are first selectively amplified, e.g. by means of PCR. In PCR, a PCR master mix is added, and various temperature levels are repeatedly used to exponentially increase the amount of nucleic acids. The PCR master mix typically contains a buffer solution, nucleotides, polymerase, primers, magnesium chloride, and optionally bovine serum albumin (BSA). This is followed e.g. by detection of the amplified target cell nucleic acids by hybridization on a microarray.

Implementation of the concept of processing in a microfluidic system by thermal pretreatment of the sample is advantageous in that the method 100 can be carried out in a microfluidic system in a particularly specified and reproducible manner, as the temperature, the volumes, and, when the method is carried out in flow-through mode, the flow rates can be set in a particularly precise manner. Moreover, the risk of contamination of the sample externally or from the environment of the sample is minimized, as the method 100 is carried out in the device 200 as a closed system.

FIG. 6 shows a device 200 for processing a sample of biological material containing target cells and companion cells in order to extract nucleic acids of the target cells according to an exemplary embodiment of the present invention. The components of the device 200 shown in the figure are a sample chamber 210, a filter 240, a waste material chamber or capture chamber 250 for lysate and wash buffer, a lysis buffer storage chamber 260, a collecting chamber or eluate capture chamber 270, three membranes 620 shown merely as examples of a separation filter, a lysis chamber 630 for collecting filtrate, a binding buffer storage chamber 662, a wash buffer storage chamber 664, and an elution buffer storage chamber 666. In this case, the device 200 is configured as a microfluidic system or a part of a microfluidic system. Preparation is carried out using the three membranes 620 shown only as examples for three-time filtration of the sample.

Three membranes 620 are arranged in a row between the sample chamber 210 and the lysis chamber 630. The sample chamber 210 is connected by means of a fluid connection via the three membranes 620 to the lysis chamber 630. The lysis buffer storage chamber 260 and the binding buffer storage chamber 662 are connected by means of a fluid connection to the lysis chamber 630. The filter 240 is arranged between the lysis chamber 630 on the one hand and the capture chamber 250 for lysate and wash buffer and the eluate capture chamber 270 on the other. The lysis chamber 630 is therefore arranged between the membranes 620 on the one hand and the filter 240 on the other.

The wash buffer storage chamber 664 and the elution buffer storage chamber 666 are connected so as to conduct fluid to a fluid connection between the lysis chamber 630 and the filter 240. Operation of the device 200 will be discussed below.

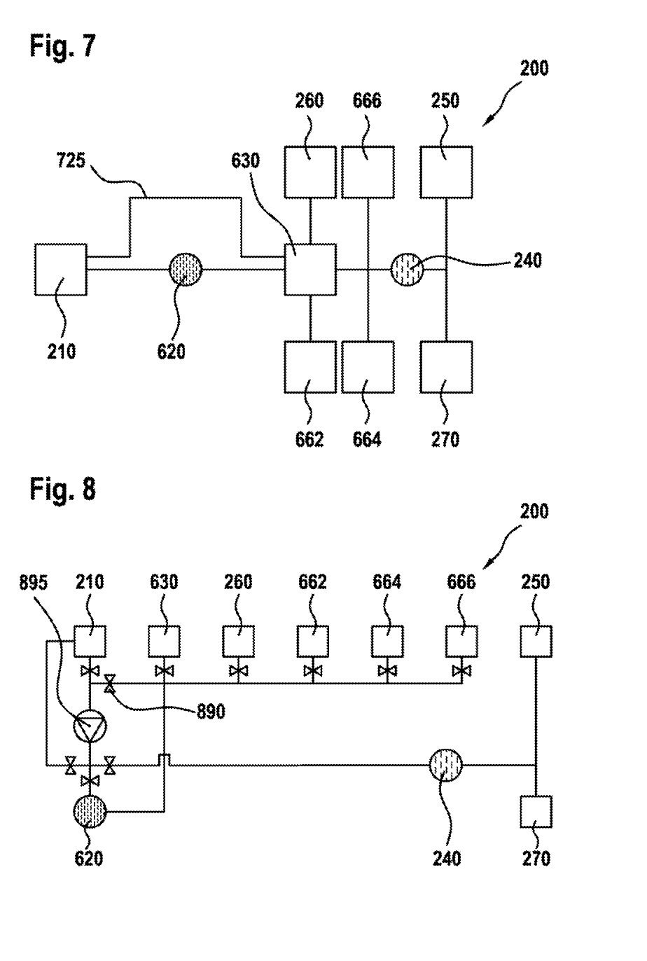

FIG. 7 shows a device 200 for processing a sample of biological material containing target cells and companion cells in order to extract nucleic acids of the target cells according to an exemplary embodiment of the present invention. In this case, the device 200 shown in FIG. 7 corresponds to the device of FIG. 6, except that only one membrane 620 is provided, and a return duct 725 is additionally arranged between the lysis chamber 630 and the sample chamber 210. In this case, the return duct 725 is used in order to repeatedly pump the sample in a circuit through the membrane 620. This is advantageous in that only one membrane 620 is needed. Operation of the device 200 will be discussed below.

FIG. 8 shows a device 200 for processing a sample of biological material containing target cells and companion cells in order to extract nucleic acids of the target cells according to an exemplary embodiment of the present invention. The device 200 in FIG. 8 corresponds to the device of FIG. 7, except that a plurality of microvalves 890 for liquid control, a pump 895, and an interconnection of chambers deviating slightly from the device of FIG. 7 are provided. In this case, the sample is pumped several times from the sample chamber 210 by means of the pump 895 through the membrane 620 into the lysis chamber 630 and then back to the sample chamber 2. Lysis or decomposition takes place in the sample chamber 210 by pumping additional lysis buffer in from the lysis buffer storage chamber 260. Operation of the device 200 is discussed in further detail below.

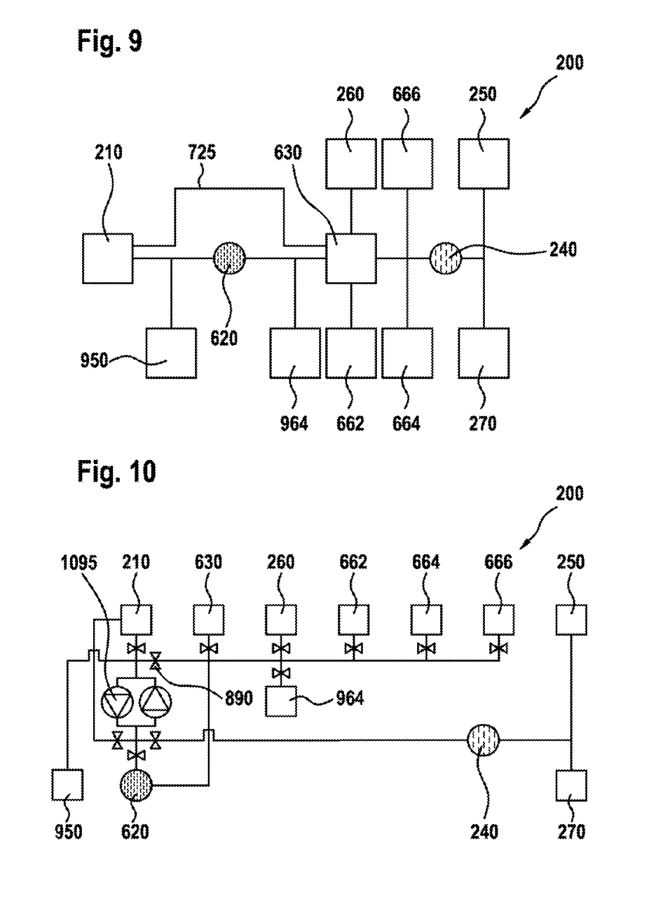

FIG. 9 shows a device 200 for processing a sample of biological material containing target cells and companion cells in order to extract nucleic acids of the target cells according to an exemplary embodiment of the present invention. The device 200 in FIG. 9 corresponds to the device of FIG. 7, except that in addition to a waste material chamber 950, a further wash buffer storage chamber 964 is shown. The further wash buffer storage chamber 964 is connected so as to conduct fluid with a fluid connection between the sample chamber 210 and the membrane 620. The waste material chamber 950 is connected so as to conduct fluid with a fluid connection between the membrane 620 and the lysis chamber 630. In this case, after each filtration, a wash buffer is pumped in the opposite direction from the further wash buffer storage chamber 964 through the membrane 620 into the waste material chamber 950. This regenerates the membrane 620, i.e. the accumulated companion cells are flushed away. Operation of the device 200 will be discussed below.

FIG. 10 shows a device 200 for processing a sample of biological material containing target cells and companion cells in order to extract nucleic acids of the target cells according to an exemplary embodiment of the present invention. The device 200 in FIG. 10 corresponds to the device of FIG. 9, except that additional microvalves 890 and a directionally reversible pump 1095 are shown. Such a configuration makes it possible by means of the pump 1095 whose pumping direction can be reversed to wash the membrane 620 between filtration steps. Operation of the device 200 will be discussed below.

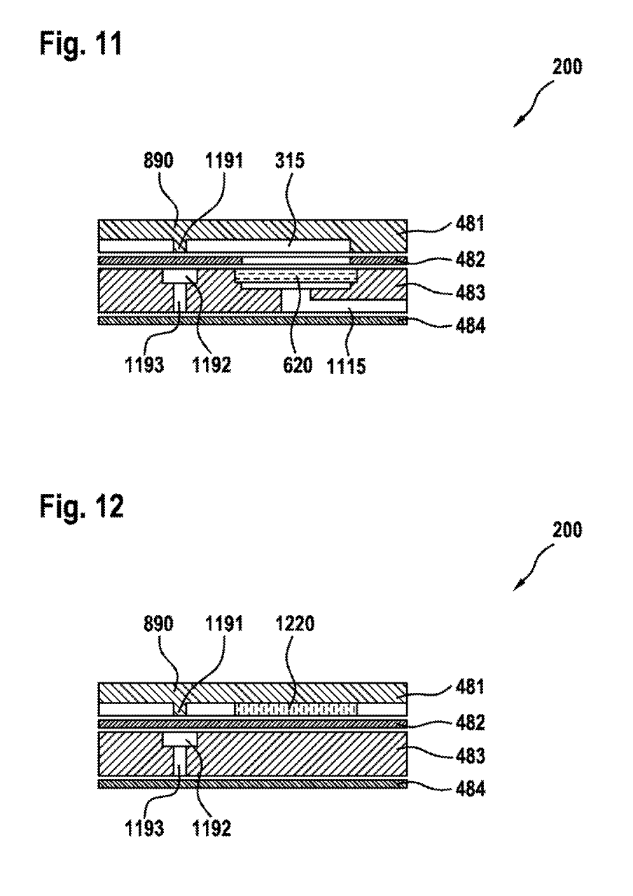

FIG. 11 shows a device 200 for processing a sample of biological material containing target cells and companion cells in order to extract nucleic acids of the target cells according to an exemplary embodiment of the present invention. The device 200 is similar to the device of FIG. 4, FIG. 8, or FIG. 10, with FIG. 11 showing a sectional view of the device 200 in an embodiment as a multilayer structure. The components of the device 200 shown are a microduct 315 as an inlet channel, the first structured polymer layer 481, the polymer film or intermediate layer 482, the second structured polymer layer 483, the polymer covering film or covering layer 484, the membrane 620, one of the valves 890, a discharge duct 1115, a projection 1191, a valve chamber 1192, and a control duct 1193.

The membrane 620 is arranged between the microduct 315 and the discharge duct 1115. The valve 890 comprises a projection 1191, the chamber 1192, in which the intermediate layer 482 is flexible, and the control duct 1193. The first structured polymer layer 481 has e.g. a thickness of 0.1 to 25 mm. The intermediate layer 482 has e.g. a thickness of 0.01 to 1 mm. The second structured polymer layer 483 has e.g. a thickness of 0.1 to 25 mm. The covering layer 484 has e.g. a thickness of 0.01 to 1 mm.

FIG. 12 shows a device 200 for processing a sample of biological material containing target cells and companion cells in order to extract nucleic acids of the target cells according to an exemplary embodiment of the present invention. In this case, the device 200 is equivalent to the device of FIG. 11, except that the device 200 in FIG. 12, instead of the membrane 620, comprises a filter duct 1220 or microduct with post structures or posts for filtering off the companion cells. The filter duct 1220 e.g. is configured in the first structured polymer layer 481.

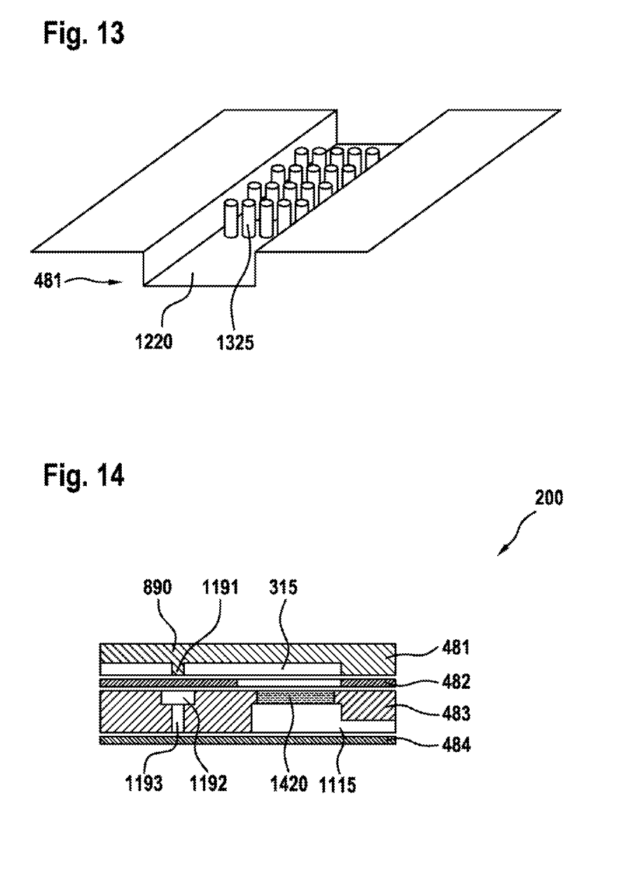

FIG. 13 shows a section of the first structured polymer layer 481 of the device of FIG. 12. It can be seen that the filter duct 1220 or microduct with post structures or posts 1325 is formed in the first structured polymer layer 481. The post structures or posts 1325 have e.g. a cylindrical shape. The width of the filter duct 1220 is e.g. between 100 μm and 10 mm, typically 1 mm. The length of the filter duct 1220 is e.g. between 1 mm and 25 mm, typically 5 mm. The depth of the filter duct 1220 is e.g. between 50 μm and 5 mm, typically 500 μm. The width of the individual posts 1325 is e.g. between 50 μm and 1 mm, typically 200 μm. The distance between the individual posts 1325 is e.g. between 5 μm and 20 μm and typically 10 μm.

FIG. 14 shows a device 200 for processing a sample of biological material containing target cells and companion cells in order to extract nucleic acids of the target cells according to an exemplary embodiment of the present invention. In this case, the device 200 is equivalent to the device of FIG. 12, with the exception that the device 200 in FIG. 14, instead of the filter duct, has a microduct with post structures or posts in the first structured polymer layer 481 and a microsieve 1420 configured in the second structured polymer layer 483. The microsieve 1420 is configured so as to filter off the companion cells.

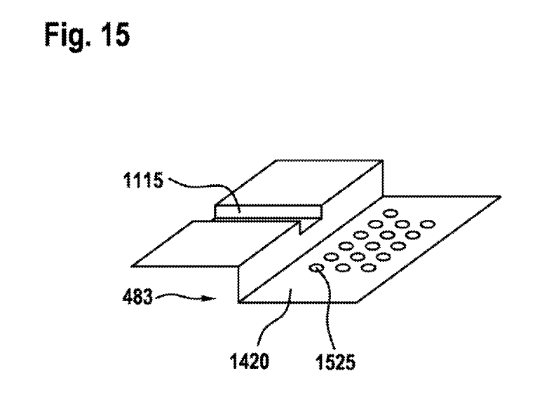

FIG. 15 shows a section of the second structured polymer layer 483 of the device of FIG. 14. In the second structured polymer layer 483, the discharge duct 1115 and the microsieve 1420 are configured with a plurality of pores 1525. The diameter of the individual pores 1525 is e.g. between 5 μm and 25 μm, and typically 10 μm.

In the following, with particular reference to FIGS. 1 and 6 to 15, a concept is presented for the preparation of a sample by means of a membrane using the method 100 and the device 200 according to exemplary embodiments of the present invention.

Specifically, in the accumulation step 110, a blood sample having a volume of between 100 μL and 10 mL is fed through the membrane 620 or the microstructures 1220 or 1420, and the filtrate produced is collected. For example, a sieve-like membrane 620 is used for this purpose, e.g. a membrane with a thickness of between 100 and 1000 μm and a pore diameter between 5 and 15 μm, e.g. between 7 and 10 μm. The sample can be fed through the membrane 620, e.g. by means of a pump 895, 1095 or by pumping using a syringe, a syringe pump, a peristaltic pump, or a membrane pump or by applying positive pressure, e.g. between 100 mb and 2 bar, for example 500 mb. Alternatively, the sample can also be fed through the membrane 620 by centrifugal forces. In the accumulation step 110, the companion cells contained in the sample are retained because of their size by the membrane 620 or the microstructures 1220 or 1420. Other components of the sample and the target cells, because of their smaller size, can pass through the membrane 620 or the microstructures 1220 or 1420 and are contained in the filtrate.

In the decomposition step 120, the cells contained in the filtrate, in particular the target cells, are decomposed or lysed. This is carried out e.g. by adding a lysis buffer to the filtrate. This lysis buffer can e.g. contain enzymes such as proteinase K, proteases and lysozyme. These enzymes destroy the cell walls of the target cells and thus release the target cell nucleic acids. The cell wall of the target cells can also be destroyed in another manner, e.g. by adding chemical reagents, e.g. chaotropic salts, detergents such as saponins, SDS or the like, or basic components such as NaOH. The liquid produced in the decomposition step 120 is referred to as lysate and contains the target cell nucleic acids, e.g. pathogenic DNA.

In the purification step 130, the target cell nucleic acids contained in the lysate are purified. This can be carried out e.g. by adsorption to a solid phase, e.g. a filter such as a silica filter, or beads such as silica beads. For example, the following process is carried out in purification on the filter 240. The lysate is mixed with a binding buffer, e.g. ethanol, and the mixture is flushed through the filter 240. The binding buffer adjusts the chemical conditions so that the target cell nucleic acids are adsorbed to the filter. This is followed by flushing a wash buffer, such as a wash buffer containing ethanol, through the filter 240. This causes cell debris and proteins to be washed off the filter 240. The target cell nucleic acids remain adsorbed to the filter 240. If needed, flushing with the wash buffer can also be repeated several times, which increases the washing effect. Finally, an elution buffer such as water is flushed through the filter 240. In this case, the target cell nucleic acids are washed off the filter 240. The target cell nucleic acids are then available in the elution buffer in a high concentration and purity.

The purification step 130 is typically followed by further steps for analysis of the target cell nucleic acids contained in the lysate. The purpose of the tests is e.g. to detect the presence of specified pathogens and resistance genes. For this purpose, the target cell nucleic acids are typically first selectively amplified, e.g. by means of the polymerase chain reaction. Detection can then be carried out, for example, by hybridization on a microarray.

According to an exemplary embodiment, in the accumulation step 110, the sample is flushed through a plurality of membranes 620, e.g. two to five membranes 620. In the individual flushing steps, a separation efficiency of less than 80%, and sometimes only 40 to 50%, is achieved. This multiple filtering increases separation efficiency. This reduces the number of companion cells contained in the filtrate and thus the amount of the companion cell nucleic acids.

According to an exemplary embodiment, in the accumulation step 110, instead of using a plurality of membranes 620 for multiple filtration, the sample or the filtrate can also be flushed several times through the same membrane 620, e.g. two to five times. This is advantageous in that the structure is simplified and only one membrane 620 is needed.

According to an exemplary embodiment, in the accumulation step 110, in cases where only one membrane 620 is used for multiple filtration, the membrane 620 is preferably washed between filtration steps in order to remove the accumulated companion cells from the membrane 620. This is advantageous in that increased flow resistance of the membrane 620 due to the accumulation of companion cells on the membrane 620 is again reduced by washing off the companion cells between filtration steps, so that subsequent filtration can be carried out in a simple and more rapid manner, i.e. at lower pressures and higher flow rates. Moreover, this is advantageous in that when large amounts of companion cells are present on the membrane 620, the risk that companion cells could detach from the membrane 620 and get into the filtrate is reduced. This increases the achievable separation efficiency. By washing the filter 620, the number of companion cells on the membrane 620 is reduced, and separation efficiency is thus increased. Washing can be carried out e.g. by flushing water or a buffer in the opposite direction through the filter 620.

According to an exemplary embodiment, in the decomposition step 120, lysis is additionally or alternatively carried out by means of ultrasound. The pressure waves and cavitation caused by the ultrasound cause the cell walls of the target cells to be destroyed in a particularly reliable and rapid manner. If necessary, the addition of a lysis buffer can also be dispensed with in this case.

According to an exemplary embodiment, in the accumulation step 110, instead of the membrane 620 or filter membrane, sieve-like microstructures 1325, 1525 are placed in channels and/or chambers of the microfluidic system. For example, these can be staggered posts 1325 with a diameter of 5 to 50 μm that are arranged at intervals of to 15 μm in the filter duct 1220. These can be manufactured, for example, in the same production step as the other structures of the microfluidic system using a replication process such as hot stamping, thus simplifying production, as separate steps for integration of the filter membrane 620 can be dispensed with. Alternatively, such a membrane with the described pore diameter can be molded by means of such a method directly on the bottom of a microfluidic duct or a chamber.

Implementing the concept of processing in a microfluidic system using a membrane is advantageous in that the method 100 can be precisely defined and reproducibly carried out in a microfluidic system, as the type of flow to the membrane 620 or the microstructures 1220 and 1420, the flow rates, and the pressures can be precisely set. Moreover, the risk of external and internal contamination of the sample or contamination from the environment of the sample can be minimized, as the method 100 is carried out in the device 200 as a closed system. When carried out in a microfluidic system, the method 100 can also be carried out in automated fashion, despite the fact that in multiple filtration, the structure is complicated, many components are required, and many work steps must be carried out.

The exemplary embodiments described and shown in the figures are selected only as examples. Different exemplary embodiments can be combined with one another either in full or with respect to individual characteristics. An exemplary embodiment can also be supplemented with features of a further exemplary embodiment. Moreover, process steps may be repeatedly listed or listed in a sequence other than the described sequence.

1. A method of processing a sample of biological material containing target cells and companion cells in order to extract nucleic acids of the target cells, comprising:
accumulating the target cells of a sample by separating the target cells or companion cells from the sample;
decomposing the target cells by at least one of chemical and physical lysis in order to produce a target cell lysate containing nucleic acids of the target cells; and
purifying the nucleic acids from the target cell lysate in order to extract nucleic acids of the target cells.

2. The method as claimed in claim 1, wherein accumulation the target cells includes:
tempering the sample to lysis temperature in order to decompose and pre-damage the companion cells;
lysing the companion cells pre-damaged in the tempering by chemical or enzymatic lysis and enzymatic digestion of nucleic acids released from the companion cells; and
separating the target cells from the sample via filtration.

3. The method as claimed in claim 2, wherein the lysing and digestion are carried out before, during, or after the tempering, with a viscosity of the sample being reduced during the lying.

4. The method as claimed in claim 1, wherein the accumulation includes separating the companion cells from the sample via filtration at least once.

5. The method as claimed in claim 1, wherein the accumulation includes flushing the sample through a plurality of separation devices connected in series in order to separate the companion cells from the sample.

6. The method as claimed in claim 1, wherein the accumulation includes:
flushing the sample through a separation device a plurality of times in order to separate lysed companion cells from the sample; and
cleaning the lysed companion cells from separation device between each flushing.

7. The method as claimed in claim 1, wherein the accumulation includes diluting the sample.

8. The method as claimed in claim 1, wherein the decomposing the target cells includes decomposing the target cells via at least one of a lysis buffer and an ultrasound coupling.

9. A device for processing a sample of biological material containing target cells and companion cells in order to extract nucleic acids of the target cells, comprising:
an accumulation device configured to accumulate target cells of a sample by separating the target cells or companion cells from the sample;
a decomposition device configured to decompose the target cells by at least one of chemical and physical lysis in order to produce a target cell lysate containing the nucleic acids of the target cells; and
a purification device configured to purify the nucleic acids from the target cell lysate in order to extract nucleic acids of the target cells.

10. The device as claimed in claim 9, wherein the accumulation device includes:
a tempering mechanism configured to temper the sample to a selection temperature for decomposing or pre-damaging the companion cells;
a storage chamber that includes a buffer solution configured to lyse companion cells decomposed or pre-damaged during tempering, by chemical or enzymatic lysis; and
an accumulation filter configured to separate the target cells from the sample.

11. The device as claimed in claim 10, the tempering mechanism is thermally coupled to a sample chamber or a sample duct arranged between a sample chamber and a decomposition chamber.

12. The device as claimed in claim 10, wherein the accumulation filter of the accumulation device is operatively connected to at least one of the decomposition device and the purification device.

13. The device as claimed in claim 9, wherein the accumulation device includes at least one separation device configured to separate the companion cells from the sample, the at least one separation device having at least one of:
a separation filter,
a filter membrane,
a filter duct with a plurality of integrated posts, and
a section provided with filter pores.

14. The device as claimed in claim 13, wherein the separation device and a return duct are arranged in parallel between the sample chamber and a decomposition chamber.

15. The device as claimed in claim 13, wherein the accumulation device includes a plurality of separation devices configured to separate the companion cells from the sample, the plurality of separation devices connected in series between the sample chamber and a decomposition chamber.
